(19) United States
(12) Patent Application Publication
(10) Pub. No.: US 2019/0133511 A1
(43) Pub. Date: May 9, 2019

(54) OCCUPANT MOTION SICKNESS SENSING

(71) Applicant: Lear Corporation, Southfield, MI (US)

(72) Inventors: Francesco MIGNECO, Saline, MI (US); David GALLAGHER, Sterling Heights, MI (US)

(21) Appl. No.: 15/808,325

(22) Filed: Nov. 9, 2017

(51) Int. Cl. A61B 5/18; A61B 5/0205; A61B 5/00; B60R 16/037; B60N 2/24; B60G 17/016; B60G 17/019; B60N 2/00

(52) U.S. Cl. CPC A61B 5/18; A61B 5/0205; A61B 5/0077; A61B 5/746; A61B 5/7278; A61B 5/02405; B60N 2/24; B60G 17/016; B60G 17/019; B60N 2/002; B60R 16/037

(57) ABSTRACT

A vehicle system is described and includes a seat configured to support an occupant and to be mounted in a vehicle and a motion sickness stimuli sensing system, which can be at least partially integrated into the seat to sense parameters experienced by a seat occupant and configured to output sensed signals, e.g., motion, oscillations, physiological parameters, an electro-dermal potential signal, and the like. A controller is configured to receive the sensed signals to determine a motion sickness of the occupant. The controller can use other sensor signals in a vehicle relating to motion sickness. The controller can output a signal to start anti-motion sickness countermeasures.

### TECHNICAL FIELD

The present disclosure relates to systems with integrated sensors to provide sensed information about the occupant to control vehicle operation, e.g., to detect motion sickness and deploy countermeasures in vehicles.

### BACKGROUND

Motion sickness is an impairing issue for many people, e.g., travelers of any age and gender. Motion sickness of an occupant in a motor vehicle can be a potentially unsafe situation and may be a cause of vehicle accidents caused by the driver. Due to its nature (dissociation between visual cue and vestibular stimuli) it is estimated to be even more relevant with the advent of autonomous vehicles.

### SUMMARY

A vehicle system with sensors to sense motion sickness-inducing stimuli that triggers motion sickness onset as experienced by a driver or occupant of the vehicle who may be seated in any vehicle seat. The seat may be configured to support an occupant and be mounted in a vehicle. Various sensors may be used to detect physiological parameters that indicate motion sickness.

A motion sickness-inducing stimuli sensing system is at least partially integrated into the seat and/or vehicle to sense stimuli (i.e., frequency, magnitude, forces, displacement, acceleration, jerk, etc.) that can induce motion sickness in a living creature and configured to output stimuli values (i.e., frequency, magnitude, forces, displacement, acceleration, jerk measurements and the like). A controller is positioned in the vehicle to receive such measurements to determine presence of the motion sickness-inducing stimuli.

An electro-dermal potential sensing system is at least partially integrated into the seat to sense physiological properties of an occupant and configured to output an electro-dermal potential signal. A controller is positioned in the vehicle to receive the electro-dermal potential signal from the electro-dermal potential sensing system to determine a motion sickness of the occupant.

In an example embodiment, at least one physiological parameter is one or more of heart rate, respiration rate, heart rate variability, Cardiorespiratory Coupling/Synchrogram (CRS).

In an example embodiment, the control signal is to adjust operation of a collision avoidance system or an adaptive braking system in the vehicle.

In an example embodiment, the electro-dermal potential system includes a plurality of contactless sensors mounted in the seat.

In an example embodiment, the seat includes a head rest. The plurality of contactless sensors includes one or more headrest sensors mounted in the headrest to measure electro-dermal potential at a head of the driver or other physiological parameters.

In an example embodiment, the seat includes a driver warning device to indicate to the driver that motion sickness is determined by the controller.

In an example embodiment, the controller measures the motion sickness-inducing stimuli based on frequency, magnitude, forces, displacement, acceleration, jerk etc. detected at the interface between the occupant and the vehicle (seat, steering wheel, pedals, etc.).

In an example embodiment, the motion sickness-inducing stimuli sensing system includes a plurality of sensors mounted in the seat, steering wheel, pedals, any site of contact between the occupant and the vehicle.

In an example embodiment, the controller measures motion sickness based on individual frequency components in the electro-dermal potential signal.

In an example embodiment, the controller uses the electro-dermal potential signal as an input to determine driver motion sickness and when motion sickness is detected outputs the control signal to increase a time to impact variable in an object avoidance calculation.

In an example embodiment, the controller measures motion sickness based on physiological parameters of one or more of heart rate, respiration rate, heart rate variability, CRS (Cardiorespiratory Coupling/Synchrogram).

In an example, embodiment, the motion sickness stimuli sensing system includes sensors at least partially integrated into the seat, a vehicle cabin, or both and adapted to sense at least one of frequency, magnitude, forces, displacement, acceleration, jerk, or combinations thereof.

In an example, embodiment, the vehicle includes and an active suspension system that is adjusted to reduce offending forces to assist the occupant to overcome the motion sickness.

In an example, embodiment, the ride profile of the seating system is adjusted to assist the occupant to overcome the motion sickness.

In an example embodiment, the sensor signals can include a video output from a cabin camera to detect physiological parameters of the occupant. The controller can use the video output and the electro-dermal potential signal to determine motion sickness state of the occupant.

A vehicle system is described that include a vehicle safety sensor system configured to sense external objects around the vehicle and output an external sensor signal. The vehicle system may also include a seat configured to support an occupant and to be mounted in a vehicle and an electro-dermal potential system at least partially integrated into the seat and configured to output an electro-dermal potential signal. A controller is to receive the electro-dermal potential signal from the electro-dermal potential system, another sensed physiological signal and the external sensor signal and to output a control signal, using the electro-dermal potential signal and sensed physiological signal to determine motion sickness. The controller can adjust operation of the vehicle safety sensor system in the vehicle based on motion sickness determination and the external sensor signal. In an example embodiment, the electro-dermal potential system includes a plurality of contactless sensors mounted in the seat of the vehicle. In an example embodiment, the sensed physiological signal can be produced by sensors in the vehicle cabin or in the vehicle seat.

Any of the above examples may be combined with each other to form additional embodiments of the present disclosure.

### BRIEF DESCRIPTION OF THE DRAWINGS

FIG. 1 is a schematic view of a vehicle according to an example embodiment.

FIG. 2 is a schematic view of a vehicle seat with sensors therein according to an example embodiment.

FIG. 3 is a process flow for determining motion sickness according to an example embodiment.

FIG. 4 is a process flow for determining motion sickness according to an example embodiment.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The present disclosure is generally directed to a seat sensors array that can be embedded in any part of the foam, trim, frame, headrest or a combination thereof of a vehicle seat. The seat sensors can be contactless sensors, which produce sensed signals, which may be combined with other sensed signals, through the acquisition of the appropriate motion sickness-inducing stimuli, physiological metrics to determine if the seat occupant is experiencing motion sickness. This detection or determination of motion-sickness can be ahead of full-blown motion sickness symptoms, which in severe cases may include nausea and vomiting. The vehicle or seating system may employ motion sickness countermeasure treatments upon detection of the onset of motion sickness. The countermeasures may include non-pharmacological countermeasures, e.g., autogenic feedback training exercise, breathing control, non-invasive vagal maneuvers, and the like. The present motion detection and countermeasure can be used in any seat in a vehicle.

A number of sensors are placed in any site of contact between the occupant and the vehicle. The sensors (e.g., accelerometers, force transducers and the like) are capable of detecting frequency, magnitude, force, acceleration, jerk, etc. of stimuli capable of inducing motion sickness. We can measure a wide range of vibrations but the most offending frequencies are from 0.001 Hz to 10 Hz, with a frequency of about 0.2 Hz having the greatest adverse effect on the occupant. Frequencies in that range with at least 0.1 m/sec² magnitude recorded in the fore-aft direction, (Y-axis) side-to-side motion (X-axis) and up and down direction (Z-axis) are capable of inducing motion sickness. The sensed physiological measurement or EDP brain wave can be temporally aligned with the sensed motions. The use of these two different sensed signals (vibration and physiological measurement) can predict motion sickness with a high degree of accuracy. Thus, the use of multiple different types of sensed signals can be combined in a sensor fusion effect to determine motion sickness.

A number of contactless electrodes and/or sensors are placed in proximity of the occupant's head and chest cavity, e.g., embedded in the seat. The sensors are capable of detecting Heart Rate (HR), Heart Rate Variability (HRV), Breathing Rate (BR), Cardiorespiratory Coupling/Synchrogram (CRS).

The sensors embedded in the headrest are capable of detecting EDP (e.g., EEG-like brain activity) and the individual frequency sub-components (α, β, δ, ϑ). At the onset of motion sickness, a clear and distinctive pattern will appear with an increased firing of the sympathetic system and HRV reduction (e.g., a stress-like reaction). At the same time, the power of all EDP subcomponents increase with changes to their ratios.

In an example embodiment, machine vision data is used in combination with the sensed EDP data and the physical data to help to positively identify positions and activities that tend to induce motion sickness. This can be used in the vehicle to assist the occupant in to reduce activities that induce motion sickness.

At least one of the sensors uses non-contact detection at a distance to determine the electro-dermal potential (EDP) originating primarily from cortical activity. This will reveal high-level central nervous system (CNS) functions, such as motion sickness. The systems described herein employ real-time processing of the electrical potential fluctuations, e.g., comparing various frequency bands of the sensed signal with respect to each other. These can act as the primary brain activity quantitative classifiers. The present system, through the acquisition of the appropriate physiological metrics, and use of a software algorithm, is capable of determining if the occupant is about to experience motion sickness or is experiencing motion sickness.

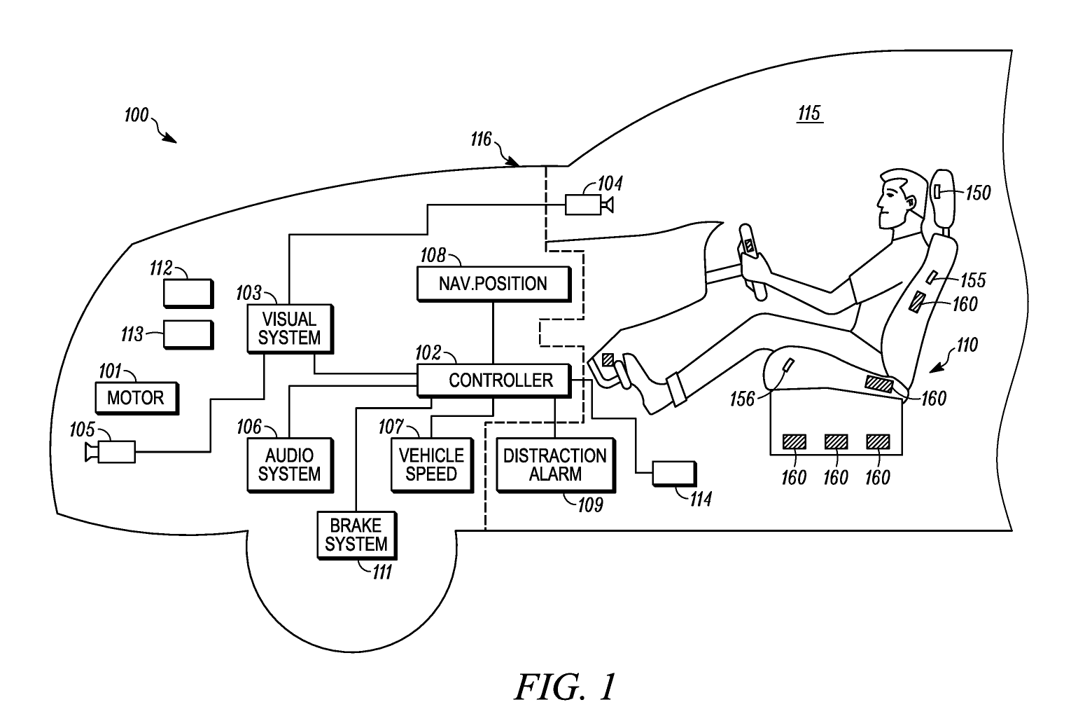

FIG. 1 shows a vehicle 100 including a cabin 115 and an engine bay 116, which can be forward of the cabin 115. The engine bay 116 houses a motor 101 that provides motive power to the vehicle. A controller 102 includes an electrical signal processor adapted to execute tasks, which can be stored in a memory. The controller 102 can be used to determine motion sickness of an occupant of a set or a vehicle. The tasks can process sensed signals according to rules loaded into the controller 102. The sensed data can be stored in memory associated with the controller 102.

Visual systems 103 are provided to receive instructions from the controller 102 and produce visual displays in the vehicle, e.g., in the cabin on display screens, the dashboard, a mobile electronic device associated with the vehicle. The displays produced by the visual systems can be images sensed by and internal camera 104, an external camera 105, collision warnings, motion sickness warnings and the like. The visual system 103 can process the image data from the cameras 104, 105 before providing the image data to the controller 102. The visual system 103 can process in images to identify objects and the position of the driver in an example embodiment. This data can be provided to the controller 102. The displays can also be anti-motion sickness images produced by the controller 102.

The internal camera 104 can sense physiological parameters of a vehicle occupant. The camera 104 can monitor the occupant's eye and/or musculoskeletal positioning. Certain eye movements or musculoskeletal positioning can be indicative of motion sickness.

An audio system 104 can be part of a head unit in the vehicle. The audio system 104 can sense audio in the cabin 115 and output audio into the cabin, e.g., using multiple speakers. The audio output from the audio system 104 can be warnings or anti-motion sickness instructions as described herein based on instruction from the controller 102. The audio output can be spoken words or tones to indicate anti-motion sickness instructions, change in settings, imminent danger, activation of collision warning system or combinations thereof.

A vehicle speed sensor 107 is provided to detect the speed of the vehicle and provide a speed signal to the controller 102. The vehicle speed can be used as a possible indicator of motion sickness. Vehicle speed can be stored when motion sickness is detected. In an example, a person may experience motion sickness at certain speeds.

A navigational position system 108 detects the position of the vehicle by receipt of satellite signals or ground based position signals. The navigational position system 108 can include a global navigation satellite system (GNSS) such as Global Positioning System (GPS), Beidou, COMPASS, Galileo, GLONASS, Indian Regional Navigational Satellite System (IRNSS), or QZSS. The navigational system can include a receiver that receives differential correction signals in North American from the FAA's WAAS system. The navigational position system 108 provides accurate position of the vehicle to the controller 102. The position of the vehicle may be used as an input for motion sickness detection. In an example, a person may experience motion sickness at certain the vehicle locations. The vehicle locations that may trigger motion sickness include, but are not limited to, hilly roadways, roads where stop and go traffic typically occurs, and the like.

An alarm 109 is positioned in the cabin. The alarm 109 can include mechanical alarms like vibration devices that can be positioned in the steering wheel or the seat. The alarm 109 can be a signal to vibrate a mobile electronic device associated with the vehicle and a passenger in the vehicle. The alarm 109 can be triggered when motion sickness is detected.

A vehicle seat 110 is position in the cabin 115 and is configured to support a person, e.g., a driver or a passenger. The seat 110 can include a plurality of sensors 150, 155, 156 to detect various biometric characteristics of the person. The sensors 150 can be contactless and can sense EDP adjacent the head of the seated person. The sensors 150, 155, and 156 can detect other biometric information.

A brake system 111 is provided to brake the wheels of the vehicle. The brake system 11 can be activated by the driver and can also be activated automatically by the controller, e.g., when motion sickness is detected and when a crash is detected as imminent or an imminent danger is detected as described herein.

A laser sensing system 112, e.g., a LIDAR, is provided. The laser sensing system 112 emits light in pulses and detects the light returned after the light reflects of object external to the vehicle 100. The laser sensing system 112 can produce a digital three-dimensional representation of the external environment around the vehicle in the direction of the light pulses. The laser sensing system 112 can perform laser scanning to produce a representation around the vehicle. The external environment can include other vehicles, signs, and other objects. The representation or individually identified objects can be provided to the controller 102 for use in the vehicle as described herein. When motion sickness is determined, the scanning range of the laser system 112 can be changed, e.g., increased.

A RADAR sensing system 113 is provided in the vehicle. The RADAR sensing system 113 emits radio frequency energy pulses and detects the returned pulses to identify objects around the vehicle or map the external environment. The representation or individually identified objects can be provided to the controller 102 for use in the vehicle as described herein. When motion sickness is determined, the scanning range of the RADAR system 112 can be changed, e.g., increased.

Other typical vehicle systems may be included in the vehicle 100 but are not illustrated for clarity of the drawings. The controller 102 may provide inputs to these other systems.

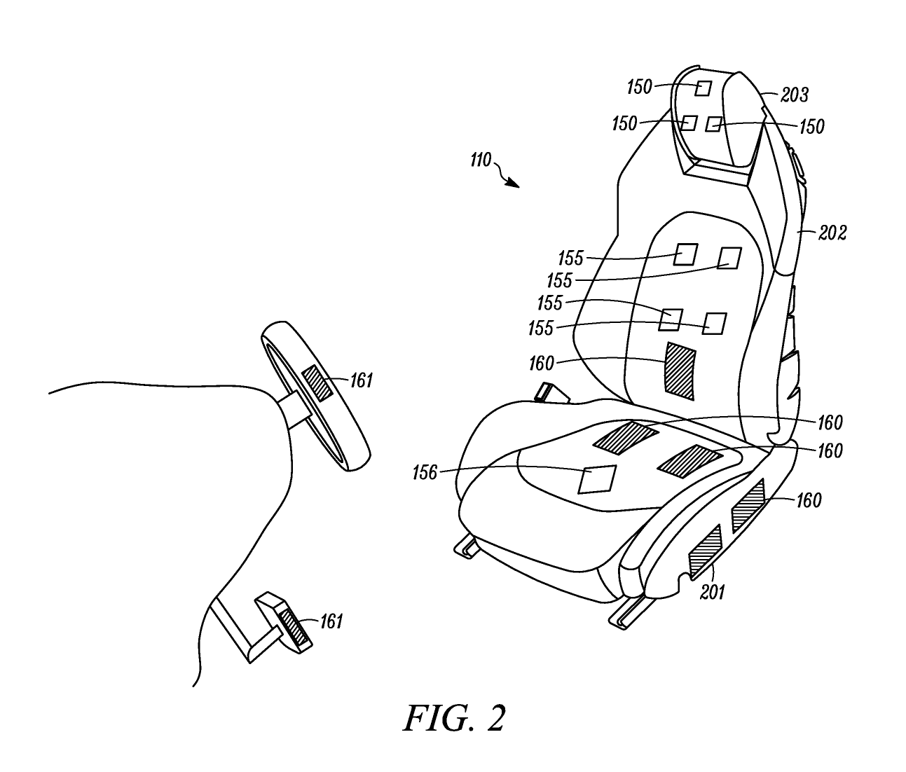

FIG. 2 shows the vehicle seat 110 configured to be fixed in a cabin of a motor vehicle. The seat 110 is adapted to support a person on a base 201 in an upright position against a seat back 202. The base 201 is fixed to the floors in the vehicle cabin, e.g., by rails. A headrest 203 may be positioned at the top of the seat back. Each of the base 201, seat back 202, and headrest 203 include a rigid frame, comfort layers on the frame and an external covering. A plurality of sensors 150, 155, 156 can be supported in the seat. A plurality of first sensors 150 may be positioned in the headrest 203 and adapted to sense EDP signals from the occupant of the seat. A plurality of second sensors 155 may be positioned in the seat back 202. The plurality of second sensors 155 may also sense EDP signals from the second occupant. The plurality of second sensors 155 may include at least one sensor that does not sense EDP signals but can sense other physiological parameters of the person. One or more third sensors 156 are positioned in the seat base 201. The third sensors 156 may also sense non-EDP signals, such as physiological parameters of the person. The plurality of second sensors 155 may include at least one sensor that does not sense EDP signals and may, e.g., sense presence of a person in the seat and sense weight of the occupant of the seat. The sensors 150 to develop raw EDP signals, which are filtered the raw signals to produce analysis signals including frequency components relevant to EDP of the person in the seat while attenuating unrelated frequency components.

In another aspect, a method is provided for monitoring a person including brain waves, e.g., EDP signals and physiological parameters. The method includes positioning a brain waves sensor adjacent the head to sense brain waves and a sensor at least proximate to portions of the skin of the body below the head to develop physiological parameters of the person. The raw sensed signals can be processed to produce at least one bandpass-filtered state-indicating signal representative of raw signal magnitude within a predetermined frequency range as an indication of the motion sickness of the person.

The sensors 150, 155 can also sense respiration rate, pulse rate, temperature, pulse volume, EDP on a limb, systolic blood pressure, diastolic blood pressure, vagal tone, coherence between respiration and heart rate, and the like. Sensors can also be positioned on the steering wheel to sense finger pulse volumes, which can be used as an input to determining motion sickness. Seat sensors 160 are positioned in the seat base or seat back. The seat sensors 160 can sense motion being experiences by the seat occupant. The seat sensors 160 can include an accelerometer, a force transducer, gyroscopes or the like. The sensors 160 can be integrated circuits or mems devices. The seat sensors 160 can detect motion in a frequency range of the 0.001 Hz to 10 Hz, with a frequency of about 0.2 Hz with at least 0.1 m/sec2 magnitude recorded in the fore-aft direction, (Y-axis) side-to-side motion (X-axis) and up and down directions (Z-axis).

Sensors 161 are mounted are in the vehicle cabin, e.g., in the control pedals, steering wheel, and the like that can be in contact with the vehicle occupant. The cabin sensors 161 can sense motion being experiences by the seat occupant. The sensors 161 be similar to the sensors 160 and can include an accelerometer, a force transducer, gyroscopes or the like. The sensors 161 can be integrated circuits or mems devices. The sensors 161 can detect motion in a frequency range of the 0.001 Hz to 10 Hz, with a frequency of about 0.2 Hz with at least 0.1 m/sec2 magnitude recorded in the fore-aft direction, (Y-axis) side-to-side motion (X-axis) and up and down directions (Z-axis). The sensors 161 can be integrated into a wearable that the occupant is wearing. Such a wearable will be in communication with the vehicle so that its sensed signals can be transmitted to the vehicle controller for processing. The sensors 161 can be part of a mobile communication device, e.g., a mobile smartphone or tablet, that is in communication with the vehicle so that its sensed signals can be transmitted to the vehicle controller for processing.

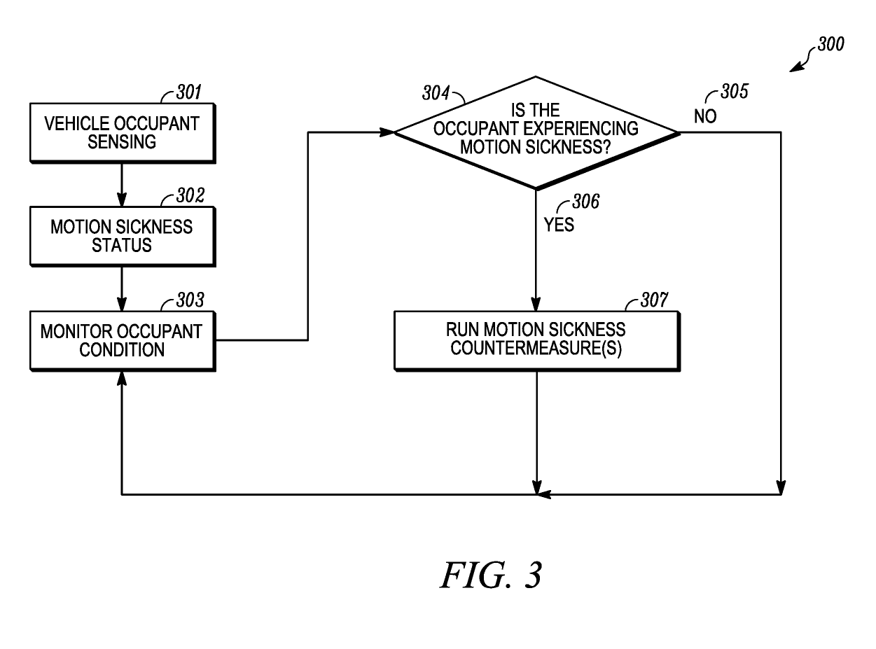

FIG. 3 shows process 300 that can be implemented in the vehicle 100 to sense possible motion sickness of the occupant of the seat. At 301, the driver or occupant is sensed in the vehicle seat. This launches a motion sickness determination algorithm at 302. The motion sickness determination algorithm 302 loads instructions from vehicle memory to controller circuitry. At 303, the seat occupant is monitored, which can include EDP sensing using the contactless sensors 150 and sensing other physiological parameters of the occupant. The use of two or more inputs, including an EDP signal and non-EDP signals, to determine motion sickness. At 304, it is determined whether the occupant is about to experience or is experiencing motion sickness. If no 305, the process returns to step 303 and the occupant is continued to be monitored. If yes 306, then the vehicle will launch motion sickness countermeasures at 307.

The driver or occupant sensed in the vehicle seat 301 also determines the individual person in the seat. Each individual may have a different motion sickness susceptibility. For example, a vehicle may be driven by multiple drivers, e.g., a husband, a wife, and a child. The individual in the seat can be identified by the sensors, e.g., by physical characteristics such as weight, height, facial recognition and the like. A key fob can also be used to identify an individual in the driver seat. The autogenic feedback therapy takes into account a stimulus response specificity, e.g., a tendency for a stimulus to evoke a consistent pattern of physiological responses from a group of individuals, and. individual response stereotypy, e.g., the tendency that an individual has to respond with the same physiological pattern.

At 303, the EDP signals are used to detect a motion sickness of the driver. The EDP signals can be separated into various sub-signals, e.g., at different frequencies, by using filters to allow certain divisions into sub-bands. These sub-bands may overlap in frequency ranges. A first sub-signal can be up to four hertz. A second sub-signal can be four hertz to seven hertz. A third sub-signal can be seven hertz to fourteen hertz. A fourth sub-signal can be fourteen hertz to about thirty hertz. A fifth sub-signal can be about thirty hertz to about one hundred hertz. Other sub-signals may overlap these ranges for the first through sixth sub-signals, e.g., from eight hertz to thirteen hertz. The relationships between these sub-signals can be used to determine whether the driver is distracted from the task of driving. The patterns of the sub-signals or the ratios of multiple sub-signals to each other can be used to determine is if motion sickness is about to start or is occurring.

At 303, a cockpit camera can be used to detect physiological parameters of the driver or occupant in the vehicle seat. The camera can detect movement or lack of movement of the driver, facial features of the driver, temperature, breathing rhythms, or combinations thereof. The camera data can be video signals sent to a data processor in the controller to determine if the physiological parameters matches a stored motion sickness pattern. Examples of motion sickness patterns can stored in vehicle memory.

The motion sickness countermeasures 307 can include at least one of autogenic feedback, neuromodulation, PEMF, active breathing control coaching or combinations thereof. The autogenic feedback can be provided by the vehicle, e.g., through the entertainment unit. The autogenic feedback can communicate to the occupant experiencing motion sickness through audible commands through the vehicle speakers and visuals shown on displays. The audible commands can encourage the occupant to perform various acts to counter the motion sickness. The monitoring of the EDP and other physiological parameters occurs while the autogenic feedback is being performed. Examples of autogenic feedback can include direction for the occupant to reduce the extrinsic stimuli, such as light and sound. The vehicle may reduce the light level in the vehicle. The sound in the vehicle can also be reduced by either reduce volume from the entertainment system or produce noise cancelation from the sound system to seemingly lower the sound level in the vehicle. The vehicle can direct the person experiencing motion sickness as determined as described herein to wear headphones that are in communication with the vehicle entertainment system. The headphones can then provide autogenic feedback to the person that is different than general cabin. The vehicle can determine which autogenic feedback works to reduce motion sickness in real time, while the occupant is in the vehicle. The autogenic feedback can include a respiration exercise, which may simultaneously teach the person to divide his/her attention. For instance, a metronome signal from the vehicle can be used to cause the person to synchronize the rate and depth of their breathing.

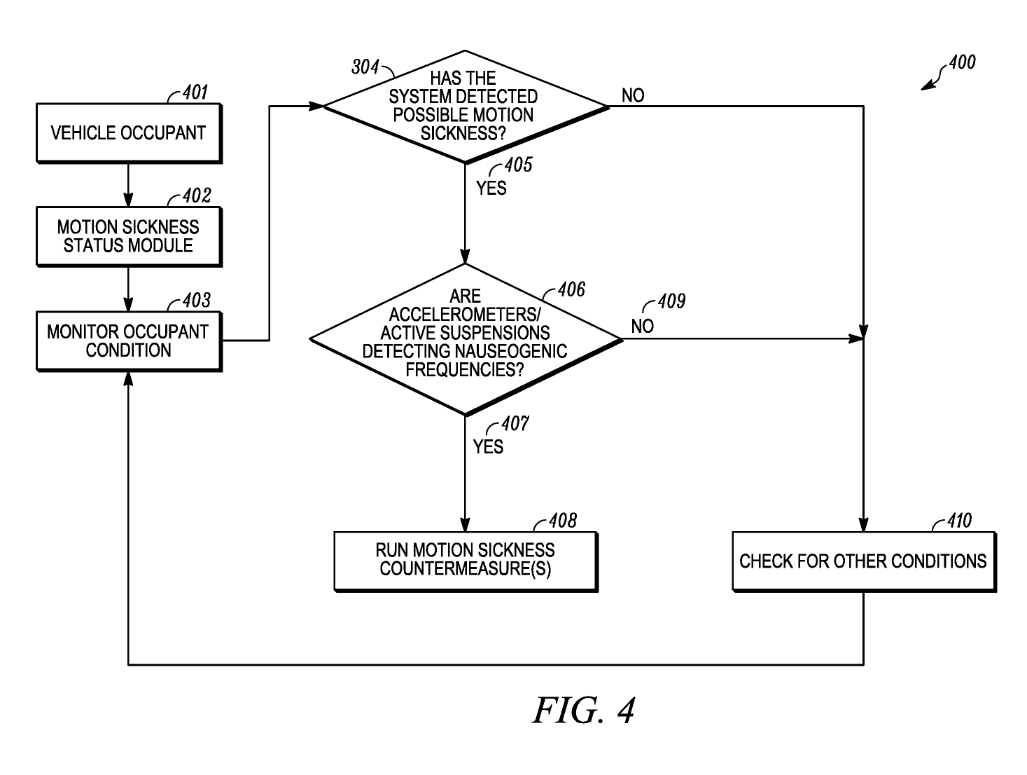

FIG. 4 shows a process 400 that can be implemented in the vehicle 100 to sense possible motion sickness of the occupant of the seat. At 401, the driver or occupant is sensed in the vehicle seat. At 401, the driver or occupant sensed in the vehicle seat determines the individual person in the seat, using the process as described with reference to step 301. This launches a motion sickness determination algorithm at 402. The motion sickness determination algorithm 402 loads instructions from vehicle memory to controller circuitry. At 403, the seat occupant is monitored, which can include EDP sensing using the contactless sensors 150 and sensing other physiological/motion sickness stimuli parameters of the occupant using other sensors 155, 160, 161. The sensing 403 can include the steps as described above with reference to step 303. The use of two or more inputs, including an EDP signal and motion sickness sensed parameters to determine motion sickness. At 404, it is determined whether the occupant is experiencing possible motion sickness or about to experience, or is experiencing motion sickness using at least two sensed signals relating to the occupant. If “NO” at 409, the process checks for other conditions, e.g., distractedness or drowsiness and returns to step 403 and the occupant is continued to be monitored. If “YES” 405, then the vehicle will launch other seat sensors to detect nauseating motions or signals at the occupant at 406. The sensors 160, 161 can be used to sense the motion being experienced by the occupant. The sensed signals can be motion signals, signals at a seat or other vehicle contact to the occupant. The motion can be array of accelerometers and/or gyroscopes and/or force transducers to measure the lateral oscillations, fore-aft oscillations, and vertical oscillations to determine frequencies, magnitude, force, relative acceleration, jerk and snap of the vehicle/seat system as a representative of that is being experienced by the occupant. The oscillation measurement by the sensors can include frequencies, relative acceleration, jerk and snap of the vehicle/seat system. The oscillations can be measured in a nauseogenic range of frequencies (0.1-10.0 Hz). These oscillations are tracked in time to correlate these with the biometric parameters to further provide data relating to motion sickness.

If the motion is a nausogenic and triggering motion sickness, then the decision 406 results in a “YES at 407 and launches motion sickness counter measures at 408.

If step 406 does not confirm motions sickness, then the process results in a NO at 409 and the process moves to a check for other occupant conditions. Other occupant conditions can include distractedness or drowsiness. Examples are described in co-pending patent application Ser. No. 15/792,085, filed Oct. 24, 2017, titled DROWSINESS DETECTION SYSTEM, which is incorporated herein in its entirety.

Embodiments of the presently described motion sickness detection may provide a specificity and a precision to the detection motion sickness and reduce the number of false positives of motion detection. The seat and/or the vehicle may include an array of accelerometers and/or gyroscopes and/or force transducers to measure the lateral motion, fore-aft motion, and vertical motion, e.g., oscillations, to determine frequencies, magnitude, force, relative acceleration, jerk and snap experienced by the occupant, e.g., using the vehicle/seat system as described herein. The seating system may include an active suspension system to determine the frequencies, relative acceleration, jerk and snap of the vehicle/seat system. The motion sickness detection methods and system can detect a nauseogenic range of frequencies (e.g., 0.01-10 Hz) and within the same time range a change in the occupant's biometric make up (e.g., brain signals, heart rate, heart rate variability, breathing rate) suggesting onset of motion sickness is detected, then a warning and/or a number of countermeasures to motion sickness can be activated.

A vehicle system is described that can include a global positioning system (e.g., GPS in North America) configured to monitor and track global positioning of the vehicle. A vehicle system is configured to share motion sickness data between infrastructure and/or other vehicles. A seat is configured to support an occupant and to be mounted in a vehicle. An electro-dermal potential system is at least partially integrated into the seat and configured to output an electro-dermal potential signal. A physiological sensor is in the seat to sense at least one physiological parameter of the occupant. A controller is configured to receive the electro-dermal potential signal from the electro-dermal potential system and the physiological parameter to determine motion sickness. The controller outputs a control signal based on determination of motion sickness. The vehicle can use the output signal to adjust operation of the vehicle safety sensor system in the vehicle. In an example embodiment, the GPS routing features may be configured to avoid routes known to induce motion sickness in a given population of drivers who have traveled a given route.

Long term data related to motion sickness detection can be processed secondary to the real-time algorithms to provide a variety of statistical information for both the occupant and machine learning systems. The long-term data may be stored in the vehicle or off-vehicle. The vehicle may include electronic communication to an external server, e.g., over WIFI, mobile communication networks, such as cellular communications, and the like. The long-term motion sickness calculations may be used to alter the instructions for determining motion sickness. The present disclosure quantifies the motion sickness status of the driver. The vehicle can use the motion sickness status of the driver to manipulate reaction times of various vehicle safety systems, e.g., the adaptive braking system, to optimize the response of the system itself. This may reduce the risk of accidents.

The present system can be used in an autonomous vehicle, e.g., a level 1-2 automobile, the vehicle needs to know the level of distraction due to motion sickness, to be able to judge the most appropriate time to switch from manual to autonomous drive and vice-versa.

This system is beneficial to all modes of transportation extending even beyond automotive and personal vehicle.

The present disclosure illustrates a controller 102. It is within the scope of the present disclosure for the controller 102 to represent multiple processors, memories and electronic control units, which can work independently with various systems to affect the functions and tasks described herein. The vehicle may use a more distributed controller system then a single controller and remain within the scope of the present disclosure. The controller 102 include circuitry to execute processing of inputs to produce an output signal.

One example of electro-dermal potential may be a type of electroencephalography (EEG), which is an electrophysiological monitoring method to record electrical activity of the brain. It is typically noninvasive, with the electrodes placed along the scalp, although invasive electrodes are sometimes used in specific applications. EEG measures voltage fluctuations resulting from ionic current within the neurons of the brain. In clinical contexts, EEG refers to the recording of the brain's spontaneous electrical activity over a period of time, as recorded from multiple electrodes placed on the scalp. Diagnostic applications generally focus on the spectral content of EEG, that is, the type of neural oscillations that can be observed in EEG signals.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A vehicle seating system, comprising:
a seat configured to support an occupant and to be mounted in a vehicle;
a motion sickness-inducing stimuli sensing system to sense stimuli capable of inducing motion sickness in the occupant and configured to output sensed stimuli values;.
an electro-dermal potential sensing system at least partially integrated into the seat to sense the occupant and configured to output an electro-dermal potential signal; and
a controller to receive the electro-dermal potential signal from the electro-dermal potential sensing system and the sensed stimuli signal to determine a motion sickness experienced by the occupant and to launch a motion sickness countermeasure for the occupant in the vehicle.

2. The vehicle seating system of claim 1, wherein the motion sickness countermeasure includes autogenic feedback to the occupant through a vehicle system.

3. The vehicle seating system of claim 1, wherein the motion sickness countermeasure includes active breathing coaching through an entertainment system of a vehicle.

4. The vehicle seating system of claim 1, wherein the physiological sensor includes a heart rate sensor to detect heart rate of the occupant.

5. The vehicle seating system of claim 1, wherein the physiological sensor includes detection of a heart rate variability, and wherein the controller detects a decrease in heart rate variability to indicate motion sickness.

6. The vehicle seating system of claim 1, wherein the physiological sensor includes detection of a breathing rate, and wherein the controller detects an increase in breathing rate to indicate motion sickness.

7. The vehicle seating system of claim 1, wherein the controller measures motion sickness based on individual frequency components in the electro-dermal potential signal.

8. The vehicle seating system of claim 7, wherein the controller measures motion sickness based on an increase in power in the individual frequency components in the electro-dermal potential signal.

9. The vehicle seating system of claim 1, wherein the controller uses the electro-dermal potential signal to determine motion sickness of the driver in the seat and when motion sickness is detected outputs the control signal to increase a time to impact variable in an object avoidance calculation.

10. The vehicle seating system of claim 1, wherein the sensor signals includes a video output from a cabin camera to detect the occupant, and wherein the controller uses the video output and the electro-dermal potential signal to determine the motion sickness of the driver.

11. The vehicle seating system of claim 1, wherein the controller outputs video signals to a display to advice the occupant to overcome motion sickness as detected.

12. The vehicle seating system of claim 1, wherein the motion sickness stimuli sensing system includes sensors at least partially integrated into the seat, a vehicle cabin, or both and adapted to sense at least one of frequency, magnitude, forces, displacement, acceleration, jerk, or combinations thereof.

13. The vehicle system of claim 1, wherein the vehicle includes and an active suspension system that is adjusted to reduce offending forces to assist the occupant to overcome the motion sickness.

14. A vehicle system, comprising:
a vehicle safety sensor system configured to sense external objects around the vehicle and output an external sensor signal;
a seat configured to support an occupant and to be mounted in a vehicle;
an electro-dermal potential system at least partially integrated into the seat and configured to output an electro-dermal potential signal;
a physiological sensor in the seat to sense at least one physiological parameter of the occupant; and
a controller to receive the electro-dermal potential signal from the electro-dermal potential system and the physiological parameter to determine motion sickness, the controller outputs a control signal based on determination of motion sickness, and the output signal adjusts operation of the vehicle safety sensor system in the vehicle.

15. The vehicle system of claim 14, wherein the electro-dermal potential system includes a plurality of contactless sensors mounted in the seat.

16. The vehicle system of claim 14, wherein the vehicle safety sensor system includes a detection and ranging system with a range setting to sense objects outside including a position and a range of an external object and the external sensor signal includes the position and range of the external object.

17. The vehicle system of claim 16, wherein the controller outputs a range extension signal when the controller determines that the driver is experiencing motion sickness, and wherein the vehicle safety system extends the range setting when the controller outputs the range extension signal.

18. The vehicle system of claim 17, further comprising a collision avoidance system having a trigger time based on the control signal from the controller, and wherein the collision avoidance system triggers an avoidance action based on the trigger time.

19. The vehicle system of claim 18, wherein the collision avoidance system has a first trigger time when motion sickness is not detected and a second trigger time when motion sickness is detected, the second trigger time being less than the first trigger time.

19. The vehicle system of claim 1, wherein the ride profile of the seating system is adjusted to assist the occupant to overcome the motion sickness

20. A vehicle system, comprising:
a vehicle safety sensor system configured to monitor and track global positioning;
a seat configured to support an occupant and to be mounted in a vehicle;
an electro-dermal potential system at least partially integrated into the seat and configured to output an electro-dermal potential signal;
a physiological sensor in the seat to sense at least one physiological parameter of the occupant; and
a controller to receive the electro-dermal potential signal from the electro-dermal potential system and the physiological parameter to determine motion sickness, the controller outputs a control signal based on determination of motion sickness, and the output signal adjusts operation of the vehicle safety sensor system in the vehicle, wherein the GPS routing features may be configured to avoid routes known to induce motion sickness of the main occupant.
